(19) United States
(12) Patent Application Publication
(10) Pub. No.: US 2021/0196208 A1
(43) Pub. Date: Jul. 1, 2021

(54) PHYSIOLOGICAL SIGNAL SENSING AND COMPENSATION SYSTEM

(71) Applicant: Industrial Technology Research Institute, Hsinchu (TW)

(72) Inventors: Shuen-Yu YU, New Taipei City (TW); Cheng-Ya LEE, Kaohsiung City (TW); Kuang-Ching FAN, Hsinchu County (TW)

(21) Appl. No.: 16/852,357

(22) Filed: Apr. 17, 2020

(30) Foreign Application Priority Data
Dec. 31, 2019 (TW) 108148577

(51) Int. Cl. A61B 5/00; A61B 5/0245; A61B 5/145; A61B 5/0428

(52) U.S. Cl. CPC A61B 5/7285; A61B 5/6801; A61B 5/0245; A61B 2562/0214; A61B 5/04284; A61B 5/7228; A61B 2560/0247; A61B 5/14517

(57) ABSTRACT

A physiological signal sensing and compensation system is provided, for contacting an object to be measured and sensing a physiological signal. The physiological signal sensing and compensation system includes a physiological signal sensing module, a compensation module, and a computing unit. The physiological signal sensing module provides an initial sensing signal. The compensation module includes a collecting mechanism for collecting a physiological liquid of the object to be measured. The compensation module provides a compensation signal according to the physiological liquid collected by the collection mechanism. The computing unit is electrically connected to the physiological signal sensing module and the compensation module, and calculates and provides a compensated sensing signal based on the initial sensing signal and the compensation signal.

### CROSS REFERENCE TO RELATED APPLICATIONS

This application claims the benefit of Taiwan Patent Application No. 108148577, filed on Dec. 31, 2019, the entirety of which is incorporated by reference herein.

### BACKGROUND

### Field of the Invention

The application relates to a physiological signal sensing and compensation system, and also relates to a system that includes a physiological signal sensing module and a compensation module.

### Description of the Related Art

Thanks to ongoing technological developments, devices that can sense human physiological signals have appeared on the market, such as wearable health bracelets, smart watches or smart headphones, or electromyography (EMG) devices, electrocardiography (ECG) devices and other devices with physiological monitoring functions. They monitor such signs as heartbeat, blood oxygen, body temperature, and heat in order to provide people with a richer experience and more accurate inspection information. However, when these physiological signal sensing devices are worn or otherwise attached to the body, the signals received by them may be different due to the devices being affected by sweat generated by human activity, which makes the obtained values or information inaccurate. To meet people's desire for lighter, more convenient and more accurate devices, how to provide a small and excellent measurement function, for example, to make a device with a sensor signal that has the same accuracy under conditions of sweating and non-sweating alike is an important subject.

### SUMMARY

An embodiment of the disclosure provides a physiological signal sensing and compensation system for contacting an object to be measured and sensing a physiological signal. The physiological signal sensing and compensation system includes a physiological signal sensing module, a compensation module, and a computing unit. The physiological signal sensing module provides an initial sensing signal. The compensation module includes a collecting mechanism for collecting a physiological liquid of the object to be measured. The compensation module provides a compensation signal according to the physiological liquid collected by the collection mechanism. The computing unit is electrically connected to the physiological signal sensing module and the compensation module, and calculates and provides a compensated sensing signal based on the initial sensing signal and the compensation signal.

### BRIEF DESCRIPTION OF DRAWINGS

The disclosure can be more fully understood by reading the subsequent detailed description and examples with references made to the accompanying drawings, wherein:

FIG. 1 is a schematic diagram showing a physiological signal sensing and compensation system according to an embodiment of the present disclosure.

FIG. 2 is schematic diagram showing a collection mechanism according to an embodiment of the present disclosure.

FIG. 3 is schematic diagram showing a collection mechanism according to another embodiment of the present disclosure.

FIG. 4 is schematic diagram showing a collection mechanism according to another embodiment of the present disclosure.

FIG. 5 is schematic diagram showing a collection mechanism according to another embodiment of the present disclosure.

FIG. 6 is schematic diagram showing a collection mechanism according to another embodiment of the present disclosure.

FIG. 7 is schematic diagram showing a collection mechanism according to another embodiment of the present disclosure.

FIG. 8 is schematic diagram showing a collection mechanism according to another embodiment of the present disclosure.

FIG. 9 is schematic diagram showing a collection mechanism according to another embodiment of the present disclosure.

FIG. 10 is schematic diagram showing an exemplary example of physiological signal spectrograms measured under different amounts of sweat; after correction of the compensation signals provided by the compensation module, those spectrograms are corrected to the same or approximately the same as a physiological signal spectrogram which is under no-sweat condition.

### DETAILED DESCRIPTION

The making and using of the embodiments of the systems and modules are discussed in detail below. It should be appreciated, however, that the embodiments provide many applicable inventive concepts that can be embodied in a wide variety of specific contexts. The specific embodiments discussed are merely illustrative of specific ways to make and use the embodiments, and do not limit the scope of the disclosure.

Unless defined otherwise, all technical and scientific terms used herein have the same meaning as commonly understood to one of ordinary skill in the art to which this disclosure belongs. It should be appreciated that each term, which is defined in a commonly used dictionary, should be interpreted as having a meaning conforming to the relative skills and the background or the context of the present disclosure, and should not be interpreted in an idealized or overly formal manner unless defined otherwise.

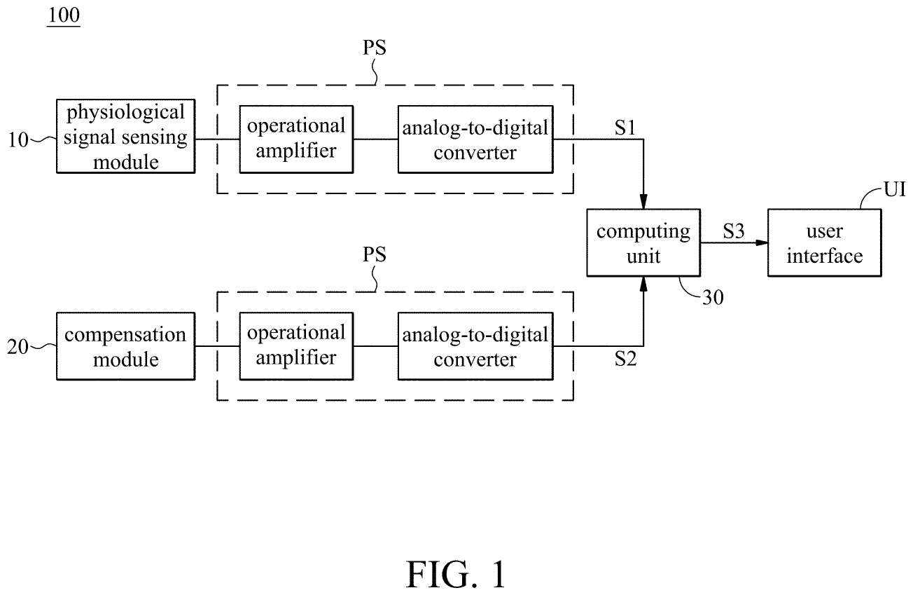

Please refer to FIG. 1, which is a schematic diagram illustrating a physiological signal sensing and compensation system 100 according to an embodiment of the present disclosure. The physiological signal sensing and compensation system 100 can be used to measure or obtain human physiological signals, such as heartbeat, electrocardiogram, myoelectricity, blood oxygen, body temperature, or heat, and can be applied to a measurement device or a wearable device. As shown in FIG. 1, the physiological signal sensing and compensation system 100 includes a physiological signal sensing module 10, a compensation module 20 and a computing unit 30. The aforementioned physiological signal sensing module 10 is configured to measure human physiological sensing information and provides an initial sensing signal S1. The compensation module 20 detects the physiological liquid or fluid (such as sweat) generated by the human body to detect the fluid volume, and then provides a compensation signal S2. The computing unit 30, may be a digital signal processor (DSP), is configured to receive the initial sensing signal S1 and the compensation signal S2 from the physiological signal sensing module 10 and the compensation module 20, and outputs a compensated sensing signal S3 according to the aforementioned signals S1 and S2.

In some embodiments, after the physiological signal sensing module 10 and the compensation module 20 obtain the initial sensing signal S1 and the compensation signal S2, the signals S1 and S2 can be transmitted to the computing unit 30 via a signal circuit module PS. The signal circuit module PS, for example, may include an operational amplifier (OP-AMP) and a signal converter, such as an analog-to-digital converter (ADC), for signal processing. In some embodiments, the aforementioned signal circuit module PS may be a part of the computing unit 30. The initial sensing signal S1 and the compensation signal S2 are transmitted to the signal circuit module PS and then to the computing unit 30 for computing. In some embodiments, the compensated sensing signal S3 may be provided to a user interface UI, so that the user can read the value. The aforementioned physiological signal sensing and compensation system 100 will be described in detail below.

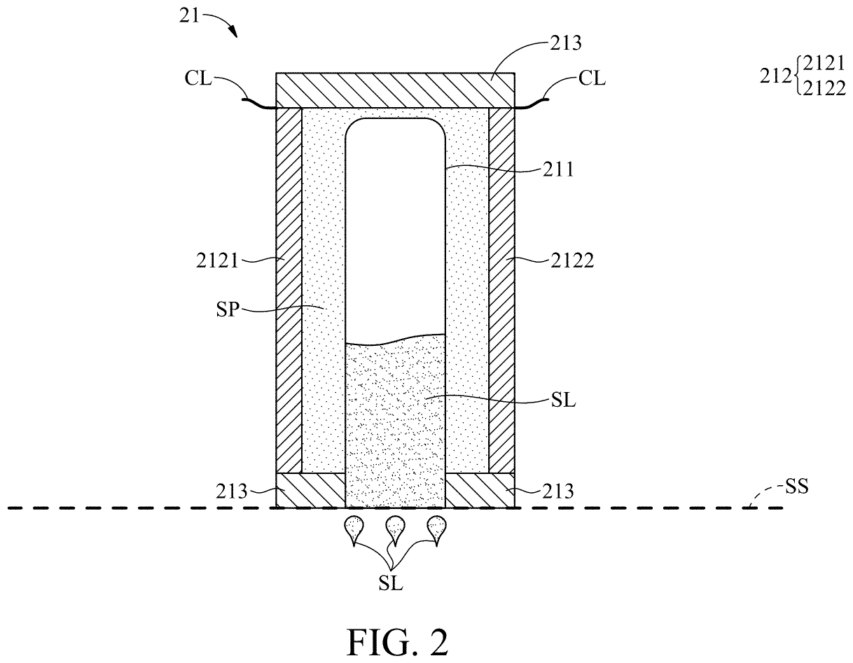

The physiological signal sensing module 10 and the compensation module 20 of the physiological signal sensing and compensation system 100 may be disposed adjacent to each other, or the compensation module 20 may be disposed on the physiological signal sensing module 10, or use the aforementioned individual configuration and set them on a sensing board to contact an object to be measured or an analyte (such as the skin surface SS in FIG. 2), for sensing the physiological signal. As shown in FIG. 2, the compensation module 20 includes a collection mechanism 21 for collecting human physiological fluids, such as sweat. In this embodiment, the collection mechanism 21 includes a collecting member 211, a capacitance element (or capacitor) 212 and a barrier member 213. The collection mechanism 21 is used to contact the human body (such as skin) SS to obtain a physiological liquid, and then the compensation module 20 can provide a compensation signal S2.

The collecting member 211 may be a pipe for collecting liquid. The capacitance element 212 has a pair of parallel plates 2121 and 2122, such as copper sheets, which are electronic components that can store electric energy in an electric field. There is a distance between the parallel plates 2121 and 2122. In some embodiments, the distance between the two is 0.5 mm to 2.0 mm. In another embodiment, the distance between the two is 1.2 mm. The collecting member 211 is disposed in the capacitance element 212 (or between the parallel plates 2121 and 2122), and a dielectric SP is filled between the parallel plates 2121 and 2122 and surrounds or compasses the collecting member 211. The dielectric SP is used to increase the electricity storage capacity of the capacitance element 212, which may be made of glass, ceramic, oxide, or other appropriate materials.

The barrier member 213 can be an outer shell having insulating material, which is disposed on the bottom side of the capacitance element 212, to prevent the capacitance element 212 from directly contacting the skin surface SS and affecting its capacitance value. The barrier member 213 is also disposed above the capacitance element 212 and the collecting member 211, which can prevent the capacitance element 212 from in contact with external substances and affecting the capacitance value. The barrier member 213 covers at least a part of the bottom of the capacitance element 212 or is disposed under the capacitance element 212, and the collecting member 211 passes through the barrier member 213 to obtain the physiological liquid on the skin surface SS.

When there is sweat SL on the skin surface SS, the collecting member 211 of the collection mechanism 21 which directly contacts the skin surface SS is able to collect and extract the sweat SL, for example, through the capillary phenomenon. The collected sweat SL will enter the collecting member 211, so that the capacitance value of the capacitance element 212 is changed. In other words, when sweat SL is present therein, the capacitance value of the capacitance element 212 in the collection mechanism 21 is different from that when no sweat SL is present. The compensation module 20 is electrically connected to the computing unit 30 via the wire CL, and provides the compensation signal S2 (a signal indicating the change in the capacitance of the capacitor 212 in this embodiment) according to the current collected amount of sweat. In this way, after receiving the initial sensing signal Si and the compensation signal S2, the computing unit 30 can correct and compensate the initial sensing signal S1 according to a compensation correspondence table established in advance in its database (such as in memory), such as analog compensation signal values, and then provide a compensated sensing signal S3.

As a result, the physiological signal sensing and compensation system 100 can provide better and more realistic data performance. The initial sensing signal S1 is adjusted by the compensation signal S2 of the compensation module 20, in order to avoid affecting the real data performance due to the sensing module 10 being affected by human physiological fluids, so that the accuracy and credibility of the measurement device are greatly improved.

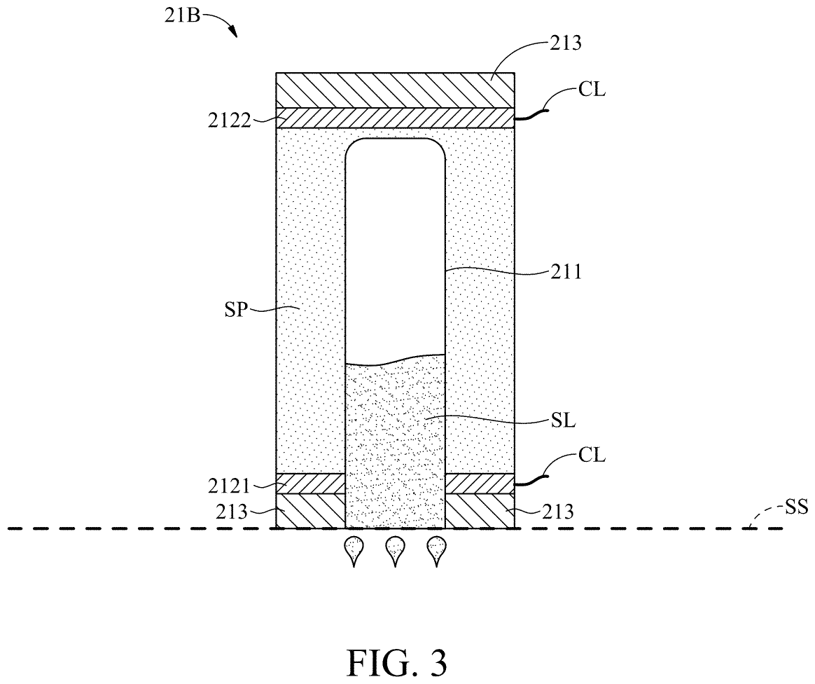

FIG. 3 shows a collection mechanism 21B according to another embodiment of the present disclosure. Compared to the collection mechanism 21 in FIG. 2, the capacitance element 212 of the collection mechanism 21B of this embodiment is arranged in a horizontal manner. That is, the parallel plates 2121 and 2122 are parallel or substantially parallel to the skin surface SS, and are perpendicular or substantially perpendicular relatively to the long axis direction of the collecting member 211. The collecting member 211 passes through the barrier member 213 and the lower parallel plate 2122 to contact the skin surface SS to collect sweat SL. The capacitance value of the capacitance element 212 is changed, and a compensation signal S2 is output to the computing unit 30. The computing unit 30 then provides a compensated sensing signal S3, so that a more real and accurate physiological information can be obtained.

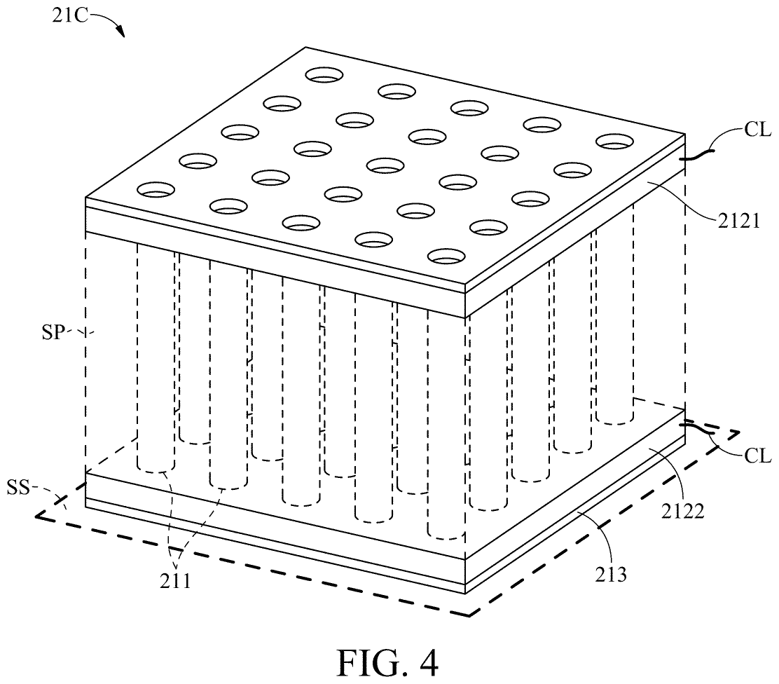

FIG. 4 shows a collection mechanism 21C according to another embodiment of the present disclosure. Compared to the collection mechanism 21B in FIG. 3, the collection mechanism 21C of this embodiment has a plurality of collecting members 211, which are arranged in the capacitance element 212 and are not connected to each other, and pass through the barrier member 213 and the lower parallel plate 2122 of the capacitance element 212, for in contact with the skin surface SS. The dielectric SP is disposed between the two parallel plates 2121 and 2122 and covers or surrounds the aforementioned plurality of collecting members 211. Through the multiple collecting members 211, a larger range of skin surface SS can be measured, and the scale of signal compensation can be made finer, so that the physiological information measured of the skin surface SS in this area is more accurate and detailed. In some embodiments, the plurality of collecting members 211 can be used as a larger collecting tank.

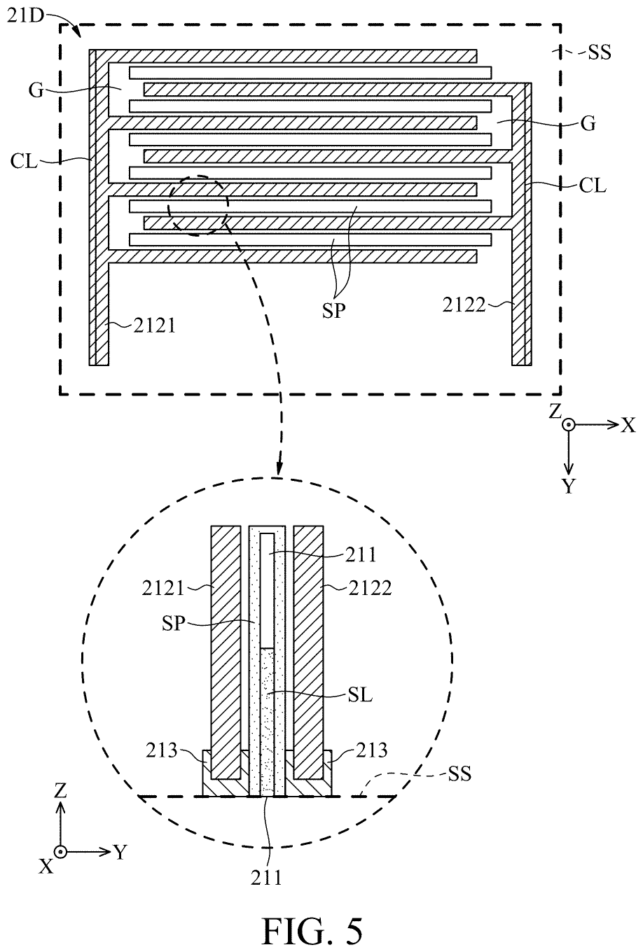

FIG. 5 shows a collection mechanism 21D according to another embodiment of the present disclosure. The capacitance element 212 of the collection mechanism 21D of this embodiment has a pair of fence-type parallel plates 2121 and 2122, and the two are arranged in a staggered manner. In detail, each fence-type parallel plate 2121 or 2122 has a plurality of parallel sub-plate, and there is a gap G between two adjacent parallel sub-plates. The gap G between two adjacent parallel sub-plates of the parallel plate 2121 and the gap G between two adjacent parallel sub-plates of the parallel plate 2122 may be the same or different. The parallel sub-plates of the fence-type parallel plates 2121 and 2122 are oppositely inserted into each other's gap G to form a plurality of pairs of parallel plates that can be filled with the dielectric SP. That is, one parallel sub-plate of the parallel plate 2122 is disposed between two adjacent parallel sub-plates of the parallel plate 2121, and the dielectric SP is disposed between the parallel sub-plate of the parallel plate 2122 and the parallel sub-plate of the parallel plate 2121 which are adjacent to each other. Similarly, one parallel sub-plate of the parallel plate 2121 is disposed between two adjacent parallel sub-plates of the parallel plate 2122, and the dielectric SP is provided between the parallel sub-plate of the parallel plate 2121 and the parallel sub-plate of the parallel plate 2122 which are adjacent to each other. In this way, the capacity of electricity storage of the capacitance element 212 can be greatly increased, the capacitance measurement area can be increased, and the measurement accuracy of the amount of sweat can be improved. This can provide a more accurate compensated sensing signal S3.

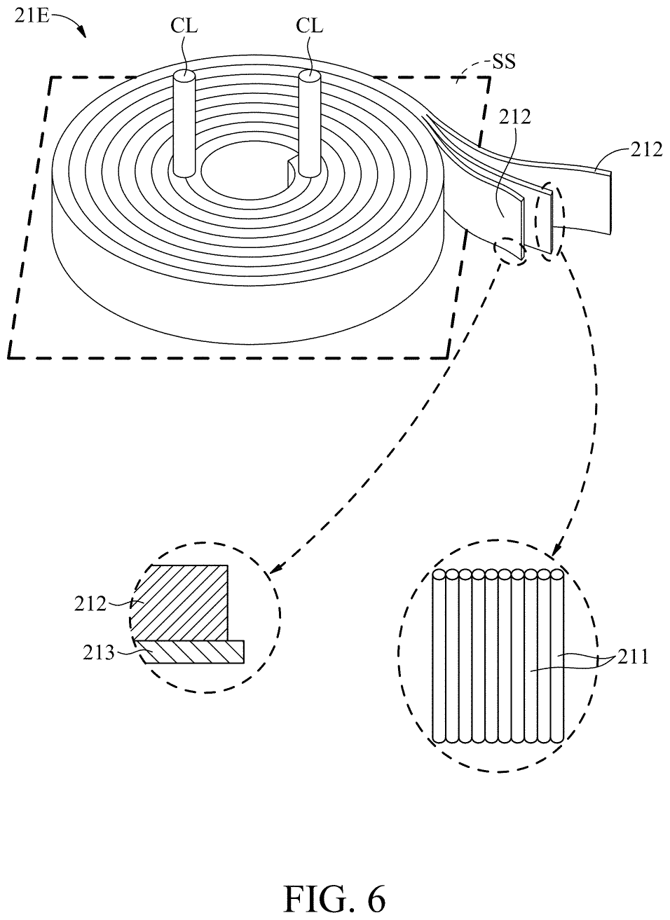

FIG. 6 shows a collection mechanism 21E according to another embodiment of the present disclosure. In this embodiment, the collection mechanism 21E is a long strip component that can be rolled into a circular structure, as shown in FIG. 6. The collection mechanism 21E includes a capacitance element 212, a plurality of collecting members 211 disposed in the capacitance element 212, and a dielectric SP (as shown in FIG. 2) filled between two parallel plates of the capacitance element 212. A barrier member 213 is disposed on the lower or bottom side of the capacitance element 212 and is used to block the direct contact between the capacitance element 212 and the skin SS, and the wire CL is disposed on the upper side (as opposed to the aforementioned lower side) of the capacitance element 212 and the collecting member 211 to electrically connect the computing unit 30 (as FIG. 1).

In the elongated collection mechanism 21E, the plurality of collecting members 211 are arranged in parallel manner, so that the collecting members 211 can be disposed on the skin surface SS at different positions, and the measurement area of the skin surface SS is increased to provide a more accurate compensation signal S2 relative to the real skin. Moreover, because the collection mechanism 21E in this embodiment is rolled up into a circular structure, the physiological measurement device applied to the human body can be significantly smaller, which benefits miniaturization.

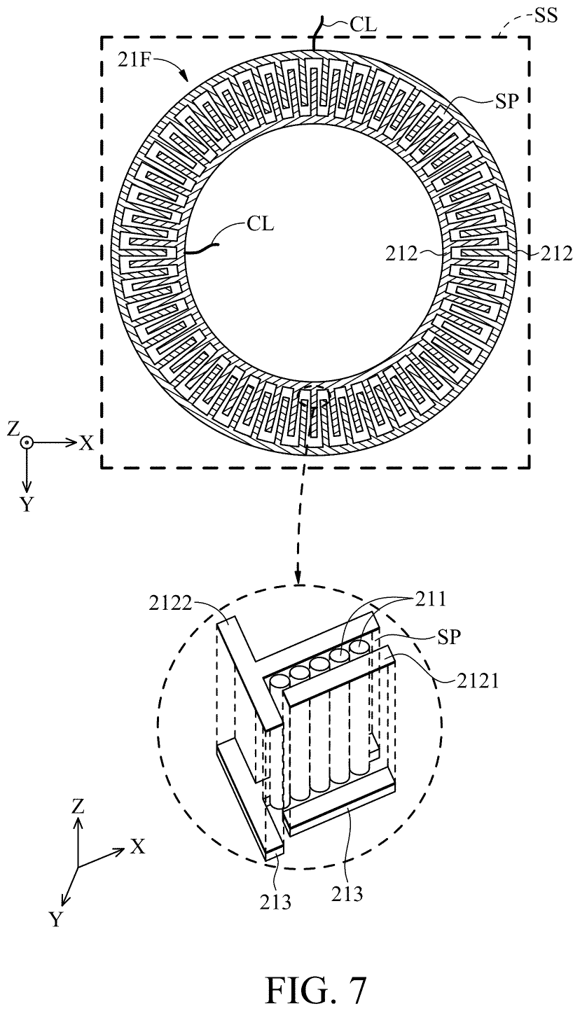

FIG. 7 shows a collection mechanism 21F according to another embodiment of the present disclosure. Compared with the fence-type collection mechanism 21D in FIG. 5, the capacitance element 212 of the collection mechanism 21F of this embodiment has an outer ring parallel plate 2121 and an inner ring parallel plate 2122. Both the outer and inner ring parallel plates 2121, 2122 have a plurality of parallel sub-plates, which is the same as or corresponding to the staggered arrangement in FIG. 5. In this way, the capacitance element 212 can provide greater electricity storage capacity, increase the capacitance measurement area, and thereby improve the measurement accuracy of the amount of sweat.

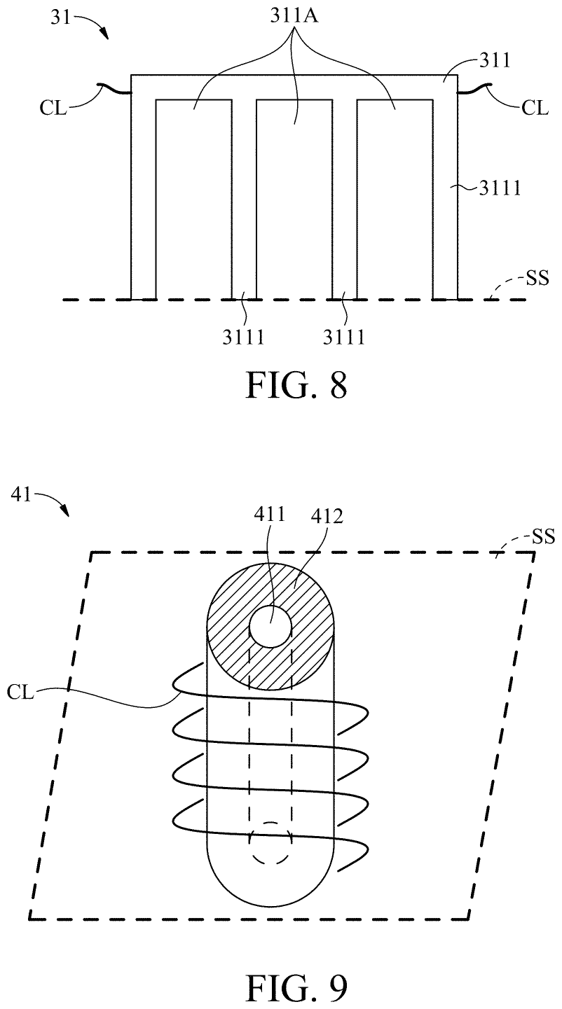

FIG. 8 shows a collection mechanism 31 according to another embodiment of the present disclosure. The collection mechanism 31 of this embodiment has a resistance element 311 having a plurality of partitions 3111 to form a plurality of intervals 311A. The wire CL is provided on both sides of the resistance element 311 to electrically connect the computing unit 30.

When the physiological signal sensing and compensation system 100 is used to sense and measure the physical signals of the human body and contact the skin surface SS, the collection mechanism 31 will be in contact with the skin surface SS, so that sweat or other physiological liquid or fluid on the surface SS can enter the interval 311A, and the equivalent resistance of the resistance element 311 is changed, the overall resistance value thereof is decreased, and the conductivity is increased. The compensation module 20 is based on the change in the resistance value of the resistance element 311 according to the liquid collected by the collection mechanism 31 to provide a compensation signal S2 which includes resistance value change.

That is, different from using the capacitance value change of the capacitance element 212 in FIGS. 1 to 7, this embodiment measures the amount of sweat according to the resistance value change of the resistance element 311, and then provides a compensation signal S2, In this way, an accurate compensation signal S2 can also be provided to the computing unit 30. The computing unit 30 then provides a compensated sensing signal S3 according to a preset resistance value change compensation table. Regarding the aforementioned resistance measurement, for example, an external power supply can be used to measure the current in series with an ammeter and to measure the voltage in parallel with voltmeter, and then the resistance value can be obtained to provide the compensation signal S2. The computing unit 30 can compare the preset resistance value according to the received resistance value information in the compensation signal S2 and obtain the difference between the two to correct and compensate the initial sensing signal S1, and then provide the compensated sensing signal S3.

FIG. 9 shows a collection mechanism 41 according to another embodiment of the present disclosure. The collection mechanism 41 in this embodiment has a collecting member 411, an inductance element 412, and a wire CL. The inductance element 412 covers or surrounds the collecting member 411, and the wire CL is wound around the outside of the inductance element 412 and is electrically connected to the aforementioned computing unit 30. When the physiological signal sensing and compensation system 100 is used to measure the physical signals of the human body and contact the skin surface SS, the collection mechanism 41 will contact the skin surface SS. If there is sweat or other physiological liquid on the surface, it will affect the inductance value of the inductance elements 412. Regarding the calculation of the inductance value, for example, the inductance value can be calculated by using the number of turns of the inductor and implementing a magnetic flux generated by a fixed amount of electricity, so as to provide a compensation signal S2 content including the change in the inductance value. That is, different from the use of capacitance or resistance changes in FIGS. 1 to 8, this embodiment measures the amount of sweat according to the change in the inductance of the inductance element 411, and then provides a compensation signal S2, which is also possible to provide an accurate compensation signal S2 to the computing unit 30. The computing unit 30 then provides a compensated sensing signal S3 according to a preset compensation value change compensation table.

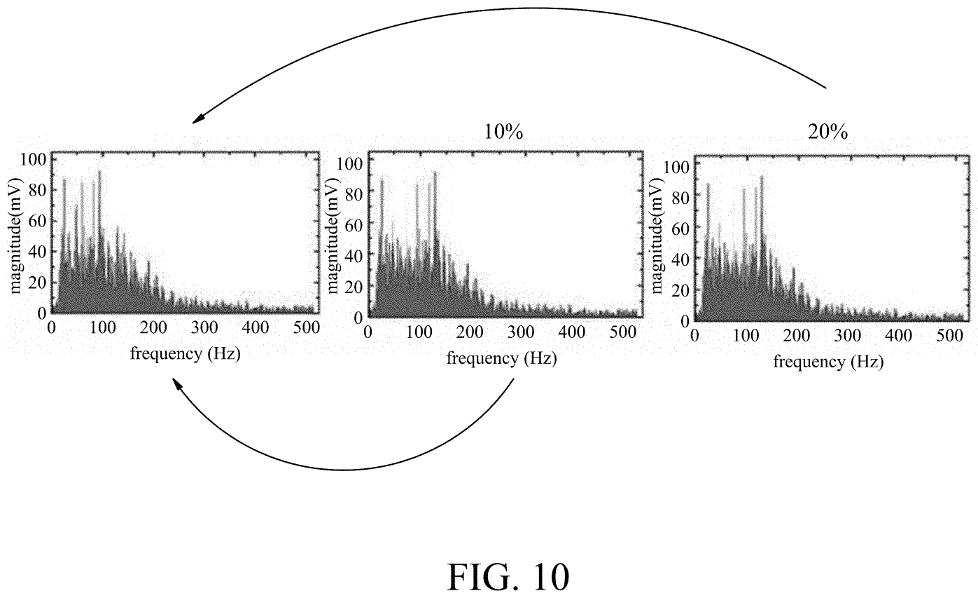

FIG. 10 shows an exemplary example of the physiological signal spectrograms measured under different amounts of sweat (including two diagrams at right side: 10% and 20%, which represent the uncompensated spectrograms of the collecting member in a state of collecting 10% and 20% of sweat, and it can be seen that sweat will affect the signal), where the horizontal axis coordinates represent the frequency, the unit is Hertz (Hz), the vertical axis coordinates indicate the relative signal magnitude in millivolts (mV), which is corrected by the compensation signal S2 provided by the aforementioned compensation module 20 (sweat collected by the aforementioned collection mechanism 21, 21B˜21F, 31, or 41), and the spectrogram of physiological signals (such as the leftmost spectrogram) can be obtained that is equal to or close to that without sweat, which can greatly improve the accuracy of information measured by the device.

As long as the features of the above embodiments do not violate the spirit of the disclosure of the present disclosure or conflict with each other, they can be mixed and used. It should be noted that the aforementioned collecting member is not limited to a long tube. In some embodiments, it may be a tank, a containing device, or a member that is appropriately filled with a physiological liquid. In some embodiments, the collected physiological liquids may also contain substances in the atmosphere, not just substances produced by the human body.

In summary, an embodiment of the present disclosure provides a physiological signal sensing and compensation system is provided, for contacting an object to be measured and sensing a physiological signal, including a physiological signal sensing module, a compensation module, and a computing unit. The physiological signal sensing module provides an initial sensing signal. The compensation module includes a collecting mechanism for collecting a physiological liquid of the object to be measured. The compensation module provides a compensation signal according to the physiological liquid collected by the collection mechanism. The computing unit is electrically connected to the physiological signal sensing module and the compensation module, and calculates and provides a compensated sensing signal based on the initial sensing signal and the compensation signal.

In the embodiment of the present disclosure, through the compensation module of the physiological signal sensing and compensation system, the liquid on the object to be collected can be collected to provide a compensation signal, and the computing unit can correct and compensate the initial sensing signal based on this compensation signal, which can improve signal accuracy, to avoid signal distortion, greatly improve the accuracy and excellence of the sensing device.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

It will be apparent to those skilled in the art that various modifications and variations can be made in the disclosure. It is intended that the standard and examples be considered as exemplary only, with the true scope of the disclosed embodiments being indicated by the following claims and their equivalents.

What is claimed is:

1. A physiological signal sensing and compensation system, configured to contact an object to be measured and sense a physiological signal, comprising:
a physiological signal sensing module, providing an initial sensing signal;
a compensation module, including:
a collecting mechanism, configured to collect a physiological liquid of the object to be measured, wherein the compensation module provides a compensation signal according to the physiological liquid collected by the collection mechanism; and
a computing unit, electrically connected to the physiological signal sensing module and the compensation module, and the computing unit calculates and provides a compensated sensing signal based on the initial sensing signal and the compensation signal.

2. The physiological signal sensing and compensation system as claimed in claim 1, wherein the compensation module is adjacent to the physiological signal sensing module.

3. The physiological signal sensing and compensation system as claimed in claim 1, wherein the collection mechanism includes a collecting member, and when the physiological signal sensing and compensation system measures a physiological signal and contacts the object to be measured, the collecting member contacts the object to be measured to collect the physiological liquid.

4. The physiological signal sensing and compensation system as claimed in claim 3, wherein the collection mechanism further includes a capacitance element, the collecting member is disposed in the capacitance element, and the capacitance element has a capacitance value;
wherein when the collecting member collects the physiological liquid, the compensation module transmits the compensation signal including the capacitance value affected by the physiological liquid to the computing unit, and the computing unit corrects and compensates the initial sensing signal according to the compensation signal, and provides the compensated sensing signal.

5. The physiological signal sensing and compensation system as claimed in claim 4, wherein the collection mechanism further includes a wire, and the wire is electrically connected to the capacitance element and the computing unit.

6. The physiological signal sensing and compensation system as claimed in claim 4, wherein the collection mechanism further includes a barrier member disposed on a lower side of the capacitance element, and when the collection mechanism contacts the object to be measured, the barrier member is located between the capacitance element and the object to be measured, and the capacitance element is not in contact with the object to be measured.

7. The physiological signal sensing and compensation system as claimed in claim 6, wherein the collecting member passes through the barrier member.

8. The physiological signal sensing and compensation system as claimed in claim 4, wherein the capacitance element includes two parallel plates, the collecting member is disposed in the capacitance element, and a dielectric is disposed between the parallel plates and surrounds the collecting member.

9. The physiological signal sensing and compensation system as claimed in claim 8, wherein the parallel plates are perpendicular to the long axis direction of the collecting member.

10. The physiological signal sensing and compensation system as claimed in claim 9, wherein the collection mechanism further includes a plurality of collecting members arranged in parallel, disposed between the parallel plates, and the collecting members are not connected to each other, and the dielectric surrounds the collecting members.

11. The physiological signal sensing and compensation system as claimed in claim 4, wherein the capacitance element includes a pair of fence-type parallel plates, and each fence-type parallel plate has a plurality of parallel sub-plates, wherein there is a gap between two adjacent parallel sub-plates of each fence-type parallel plate, and each parallel sub-plate is inserted into the gap.

12. The physiological signal sensing and compensation system as claimed in claim 11, wherein a dielectric is disposed between every two adjacent parallel sub-plates.

13. The physiological signal sensing and compensation system as claimed in claim 8, wherein the collection mechanism has a long strip shape and is rolled into a circular structure, wherein the collection mechanism has a plurality of collecting members which are located between the parallel plates.

14. The physiological signal sensing and compensation system as claimed in claim 3, wherein the collection mechanism further includes a resistance element having a plurality of partitions, there is a space between the partitions, and the resistance element has a resistance value;
wherein when the collecting member collects the physiological liquid, the compensation module transmits the compensation signal including the resistance value affected by the physiological liquid to the computing unit, and the computing unit corrects and compensates the initial sensing signal according to the compensation signal, and provides the compensated sensing signal.

15. The physiological signal sensing and compensation system as claimed in claim 3, wherein the collection mechanism further includes an inductance element and a wire, the inductance element surrounds the collecting element, the wire is wound around the outside of the inductance element and is electrically connected to the computing unit, and the inductance element has an inductance value;
wherein when the collecting member collects the physiological liquid, the compensation module transmits the compensation signal including the inductance value affected by the physiological liquid to the computing unit, and the computing unit corrects and compensates the initial sensing signal according to the compensation signal, and provides the compensated sensing signal.
